(19) United States
(12) Patent Application Publication
(10) Pub. No.: US 2026/0020990 A1
(43) Pub. Date: Jan. 22, 2026

(54) ABSORBENT ARTICLE HAVING A FRONT WAIST GUARD

(71) Applicant: The Procter & Gamble Company, Cincinnati, OH (US)

(72) Inventors: Abhishek SURUSHE, Schwalbach (DE); Lutz STELZIG, Schwalbach (DE); Udo FRIEDEL SCHOENBORN, Schwalbach (DE); Ernesto GABRIEL BIANCHI, Schwalbach (DE); Yi YUAN, Beijing (CN); Gavin Kahlil DILLAVOU-BROWN, Cincinnati, OH (US)

(21) Appl. No.: 19/344,620

(22) Filed: Sep. 30, 2025

Related U.S. Application Data
(63) Continuation of application No. PCT/CN2023/093228, filed on May 10, 2023.

(51) Int. Cl. A61F 13/494; A61F 13/49

(52) U.S. Cl. CPC A61F 13/49466; A61F 13/49009; A61F 13/49406; A61F 2013/4948

(57) ABSTRACT

An absorbent article having a front waist region, a back waist region and a crotch region extending longitudinally between the front and back waist region. The absorbent article comprises an absorbent main body with an absorbent core, and an acquisition system. A front waist guard is disposed in and attached to the front waist region such that a front pocket is formed, the front pocket extending along the longitudinal direction from a closed base line at or adjacent to the front waist edge towards an open edge.

### CROSS REFERENCE TO RELATED APPLICATION

This application is a continuation of Patent Application No. PCT/CN2023/093228, filed on May 10, 2023, which is hereby incorporated by reference herein.

### FIELD

The present invention relates to absorbent articles having a front waist guard for preventing leakage of low viscosity bodily exudates, while also maintaining other functions as an absorbent article.

### BACKGROUND

Infants and other individuals wear absorbent articles such as diapers to receive and contain urine and other body exudates. Pull-on absorbent articles, or pant-type absorbent articles are those which are donned by inserting the wearer's legs into the leg openings and sliding the article up into position about the lower torso. Pant-type absorbent articles have become popular young children but also for younger babies requiring a soft fit around the waist opening and leg openings. Moreover, pant-type absorbent articles have become popular for females having heavy flow or overnight usage during menstruation periods. One function desired for a pant-type article, as well as for absorbent articles which are applied on the wearer with tapes, is a protective measure for preventing leakage of low viscosity bodily exudates through the waist opening.

Apart from the known risk of leakage through the waist opening in the back waist region, it has now been found that leakage can also occur through the side panels in the waist region. This risk is due, as wearers do not always lie on their back or belly, but they may also lie on the side, especially while they sleep. This is, in particular, a problem for young children, who may still wear absorbent articles at night, while no longer requiring such articles during day time. However, also adults wearer absorbent articles may often sleep lying on the side. As young children and adults typically urinate larger amounts of liquid than babies, the risk of leakage through the side panels of the absorbent articles may be especially problematic.

Based on the foregoing, there is a need for an absorbent article provided with a protective measure for efficiently preventing leakage through the sides of the article's waist region.

### SUMMARY

The invention relates to an absorbent article having a longitudinal centerline and a longitudinal direction along and in parallel to the longitudinal centerline; a transverse centerline and a transverse direction along and in parallel to the transverse centerline, a front waist region with a front waist edge, a back waist region with a back waist edge and a crotch region extending longitudinally between the front and back waist region.

The absorbent article comprises an absorbent main body with longitudinally extending side edges and transversely extending end edges. The absorbent main body comprises a topsheet, a backsheet, an absorbent core, and an acquisition system, wherein the absorbent core and the acquisition system are sandwiched between the topsheet and the backsheet with the acquisiton system being provided between the topsheet and the absorbent core.

The absorbent main body further comprises elasticized barrier leg cuffs extending at least partially between the end edges along both longitudinally extending side edges of the absorbent main body in the crotch region, and extending into the front and back waist region.

The barrier leg cuffs each have a proximal edge attached to the topsheet and/or the backsheet and a free terminal edge, the free edge being intended to contact and form a seal with the wearer's skin.

A front waist guard (FWG) is disposed in and attached to the front waist region such that a front pocket is formed, the front pocket extending along the longitudinal direction from a closed base line at or adjacent to the front waist edge towards an open edge. The maximum dimension from the closed base line to the open edge parallel to the longitudinal centerline defines the front pocket length (FPL), The acquisition system is provided in the front pocket along at least 10% of the front pocket length (FPL).

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings and which like designations are used to designate substantially identical elements, and in which:

FIG. 1 is a perspective view of one embodiment of an absorbent article of the present invention.

FIG. 2 is a perspective view of another embodiment of an absorbent article of the present invention.

FIG. 3A is a schematic plan view of one embodiment of an absorbent article of the present invention with the seams unjoined and in a flat uncontracted condition showing the garment facing surface.

FIG. 3B-3C are schematic plan views of one belt embodiment of the present invention with the seams unjoined and in a flat uncontracted condition showing the body facing surface.

FIGS. 4A-4C are schematic cross section views of precursors for making the embodiment of FIGS. 3A-3C taken along line L2, with the thickness dimension exaggerated.

FIG. 4D is a schematic cross section view of the embodiment of FIGS. 3A-3C, with the thickness dimension exaggerated.

### DEFINITIONS

As used herein, the following terms shall have the meaning specified thereafter:

“Absorbent article” refers to devices that absorb and contain body exudates, particularly urine and other water-containing liquids, and, more specifically, refers to devices that are placed against or in proximity to the body of the wearer to absorb and contain the various exudates discharged from the body. Absorbent articles may include diapers (for babies and infants as well as for adult incontinence), pants (for babies and infants as well as for adult incontinence). As used herein, the term “exudates” includes, but is not limited to, urine, blood, vaginal discharges, sweat and fecal matter. Preferred absorbent articles of the present invention are disposable absorbent articles, more preferably disposable diapers, disposable pants and disposable absorbent inserts.

“Absorbent core” is used herein to refer to a structure intended to be disposed between a topsheet and backsheet of an absorbent article for absorbing and storing liquid received by the absorbent article.

“Disposable” is used in its ordinary sense to mean an article that is disposed or discarded after a limited number of usage events over varying lengths of time, for example, less than 10 events, less than 5 events, or less than 2 events. If the disposable absorbent article is a diaper, a pant, absorbent insert, sanitary napkin, sanitary pad or wet wipe for personal hygiene use, the disposable absorbent article is most often intended to be disposed after single use. The used and disposed absorbent article may or may not be subsequently recycled. As used herein, the terms ‘absorbent article’, ‘pant’ and ‘diaper’ always also refer to disposable absorbent articles, disposable pants (as well as disposable absorbent pants), and disposable diapers.

“Diaper” and “pant” refers to an absorbent article generally worn by babies, infants and incontinent persons about the lower torso so as to encircle the waist and legs of the wearer and that is specifically adapted to receive and contain urinary and fecal waste. In a pant, as used herein, the longitudinal edges of the first and second waist region are attached to each other to a pre-form waist opening and leg openings. A pant is placed in position on the wearer by inserting the wearer's legs into the leg openings and sliding the pant absorbent article into position about the wearer's lower torso. A pant may be pre-formed by any suitable technique including, but not limited to, joining together portions of the absorbent article using refastenable and/or non-refastenable bonds (e.g., seam, weld, adhesive, cohesive bond, fastener, etc.). A pant may be pre-formed anywhere along the circumference of the article (e.g., side fastened, front waist fastened). In a diaper, the waist opening and leg openings are only formed when the diaper is applied onto a wearer by (releasable) attaching the longitudinal edges of the first and second waist region to each other on both sides by a suitable fastening system.

“Belt-type pant” refers to a an absorbent article with a ring-like elastic belt comprising an absorbent main body to cover the crotch region of the wearer, a front elastic belt and a back elastic belt. The absorbent main body extends into and overlaps with the front and back elastic belt. The front and back elastic belts form a discrete elastic belt extending transversely defining the waist opening and being discontinuous in the longitudinal direction. For the belt-type pant, the discrete ring-like elastic belt may also be referred to as the elastic belt. For the belt-type pant the front and back elastic belts jointly define the waist opening and the front and back elastic belt together with the absorbent main body define the leg openings.

“Longitudinal” refers to a direction running substantially perpendicular from a waist edge to an opposing waist edge of the article and generally parallel to the maximum linear dimension of the article. “Transverse” refers to a direction perpendicular to the longitudinal direction.

“Inner” and “outer” refer respectively to the relative location of an element or a surface of an element or group of elements. “Inner” implies the element or surface is nearer to the body of the wearer during wear than some other element or surface. “Outer” implies the element or surface is more remote from the skin of the wearer during wear than some other element or surface (i.e., element or surface is more proximate to the wearer's garments that may be worn over the present article).

“Body-facing” and “garment-facing” refer respectively to the relative location of an element or a surface of an element or group of elements. “Body-facing” implies the element or surface is nearer to the wearer during wear than some other element or surface. “Garment-facing”implies the element or surface is more remote from the wearer during wear than some other element or surface (i.e., element or surface is proximate to the wearer's garments that may be worn over the disposable absorbent article).

“Film” refers to a sheet-like material wherein the length and width of the material far exceed the thickness of the material. Typically, films have a thickness of about 0.5 mm or less.

“Water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water, urine, or synthetic urine to pass through its thickness in the absence of a forcing pressure. Conversely, the term “water-impermeable” refers to a layer or a layered structure through the thickness of which liquid water, urine, or synthetic urine cannot pass in the absence of a forcing pressure (aside from natural forces such as gravity). A layer or a layered structure that is water-impermeable according to this definition may be permeable to water vapor, i.e., may be “vapor-permeable”.

“Hydrophilic” describes surfaces of substrates which are wettable by aqueous fluids (e.g., aqueous body fluids) deposited on these substrates. Hydrophilicity and wettability are typically defined in terms of contact angle and the strike-through time of the fluids, for example through a nonwoven fabric. This is discussed in detail in the American Chemical Society publication entitled “Contact Angle, Wettability and Adhesion”, edited by Robert F. Gould (Copyright 1964). A surface of a substrate is said to be wetted by a fluid (i.e., hydrophilic) when either the contact angle between the fluid and the surface is less than 90°, or when the fluid tends to spread spontaneously across the surface of the substrate, both conditions are normally co-existing. Conversely, a substrate is considered to be “hydrophobic” if the contact angle is equal to or greater than 90° and the fluid does not spread spontaneously across the surface of the fiber. The contact angle test method used for the present invention is set out herein below.

“Extendibility” and “extensible” mean that the width or length of the component in a relaxed state can be extended or increased.

“Elasticated” and “elasticized” mean that a component comprises at least a portion made of elastic material.

“Elongation rate” means the state of elongation of a material from its relaxed, original length, namely an elongation rate of 10% means an elongation resulting in 110% of its relaxed, original length.

“Elongatable material”, “extensible material”, or “stretchable material” are used interchangeably and refer to a material that, upon application of a biasing force, can stretch to an elongation rate of at least 10% (i.e. can stretch to 10 percent more than its original length), without rupture or breakage, and upon release of the applied force, shows little recovery, less than about 20% of its elongation without complete rupture or breakage as measured by EDANA method 20.2-89. In the event such an elongatable material recovers at least 40% of its elongation upon release of the applied force, the elongatable material will be considered to be “elastic” or “elastic.” For example, an elastic material that has an initial length of 100 mm can extend at least to 150 mm, and upon removal of the force retracts to a length of at least 130 mm (i.e., exhibiting a 40% recovery). In the event the material recovers less than 40% of its elongation upon release of the applied force, the elongatable material will be considered to be “non-elastic”. For example, an elongatable material that has an initial length of 100 mm can extend at least to 150 mm, and upon removal of the force retracts to a length of at least 145 mm (i.e., exhibiting a 10% recovery).

As used herein, the term “nonwoven web” refers to a material which is a manufactured web/layer of directionally or randomly oriented fibers or filaments. The fibers may be of natural or man-made origin. Natural fibers may be selected from the group consisting of wood pulp fibers, wheat straw fibers, rice straw fibers, flax fibers, bamboo fibers, cotton fibers, jute fibers, hemp fibers, sisal fibers, bagasse fibers, Hesper aloe fibers, miscanthus, marine or fresh water algae/seaweeds, silk fibers, wool fibers, and combinations thereof. Another group of fibers may also be regenerated cellulose fibers, such as viscose, Lyocell (Tencel®), rayon, modal, cellulose acetate fibers, acrylic fibers, cuprammonium rayon, regenerated protein fibers etc. Preferably, the natural fibers or modified natural fibers are selected from the group consisting of cotton fibers, bamboo fibers, viscose fibers or mixtures thereof. Preferably, the natural fibers are cotton fibers. Synthetic fibers may be selected from the group consisting of polyolefins (such as polyethylene, polypropylene or combinations and mixtures thereof), polyethylene terephthalate (PET), co PET, polylactic acid (PLA), polybutylene succinate (PBS), polyhydroxy alkanoates (PHA), nylon (or polyammide), or mixtures or combinations thereof. An alternative option is to use superabsorbent fibers, for example SAF™ which is a cross-linked terpolymer based on acrylic acid, which is partially neutralised to its sodium salt, commercially available from Technical Absorbents.

The fibers in a nonwoven web are consolidated by friction, and/or cohesion and/or adhesion, and/or by heat bonding, pressure bonding, heat and pressure bonding, and/or ultrasonic bond excluding paper and products which are woven, knitted, tufted, stitch-bonded. The fibers may be staple fibers (e.g. in carded nonwoven webs) or continuous fibers (e.g. in spunbonded or meltblown nonwoven webs).

Nonwoven webs can be formed by many processes such as meltblowing, spunlaying, solvent spinning, electrospinning, and carding, and the fibers can be consolidated, e.g. by hydroentanglement (in spunlaced nonwoven webs), air-through bonding (using hot air that is blown through the fiber layer in the thickness direction), needle-punching, one or more patterns of bonds and bond impressions created through localized compression and/or application of heat or ultrasonic energy, or a combination thereof. The fibers may, alternatively or in addition, be consolidated by use of a binder. The binder may be provided in the form of binder fibers (which are subsequently molten) or may be provided in liquid, such as a styrene butadiene binder. A liquid binder is provided to the fibers (e.g. by spraying, printing or foam application) and is subsequently cured to solidify.

The basis weight of nonwoven webs is usually expressed in grams per square meter (g/m²).

In a spunlace nonwoven web the fibers have been carded as precursor web and then subjected to hydroentanglement to intermingle and intertwine the fibers with each other. Cohesion and the interlacing of the fibers with one another may be obtained by means of a plurality of jets of water under pressure passing through a moving fleece or cloth and, like needles, causing the fibers to intermingle with one another (hereinafter also referred to as “hydraulic interlacing”). Thus, consolidation of a spunlace nonwoven web is essentially a result of hydraulic interlacing.

“Comprise,” “comprising,” and “comprises” are open ended terms, each specifies the presence of the feature that follows, e.g. a component, but does not preclude the presence of other features, e.g. elements, steps, components known in the art or disclosed herein. These terms based on the verb “comprise” encompasses the narrower terms “consisting essential of” which excludes any element, step or ingredient not mentioned which materially affect the way the feature performs its function, and the term “consisting of” which excludes any element, step, or ingredient not specified.

“Adjacent” and “adjacent to”, as used herein, means very near or in close proximity. “adjacent” may mean spaced by a distance of no more than 50 mm, or not more than 40 mm, or not more than 30 mm, or not more than 25 mm, or not more than 20 mm, or not more than 15 mm, or not more than 10 mm, or not more than 5 mm. For example, if the absorbent article is a relatively large absorbent article intended for adult incontinent wearers, “adjacent” may mean spaced by a distance of no more than 50 mm. In another example, where the absorbent article is intended to be worn by babies, “adjacent” may mean spaced by a distance of not more than 25 mm, or not more than 20 mm, or not more than 15 mm, or not more than 10 mm, or not more than 5 mm.

### DETAILED DESCRIPTION

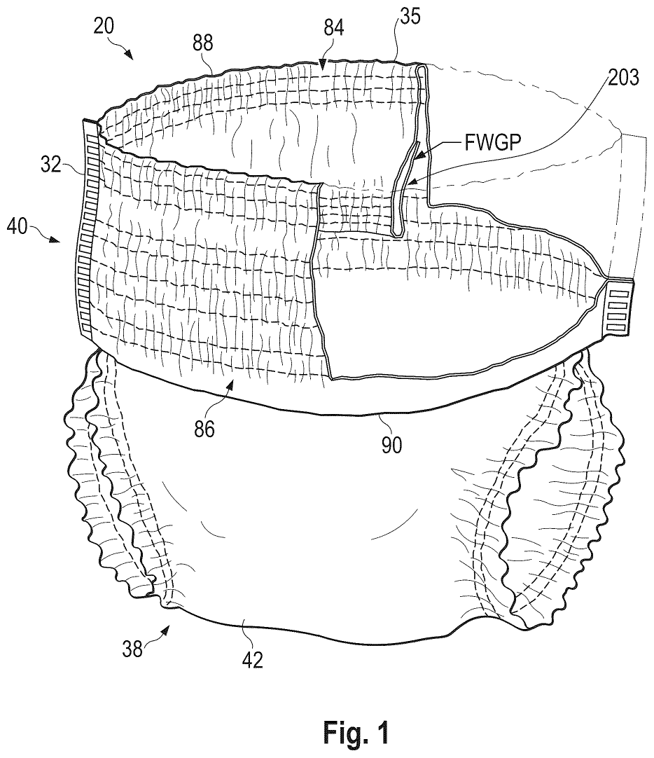

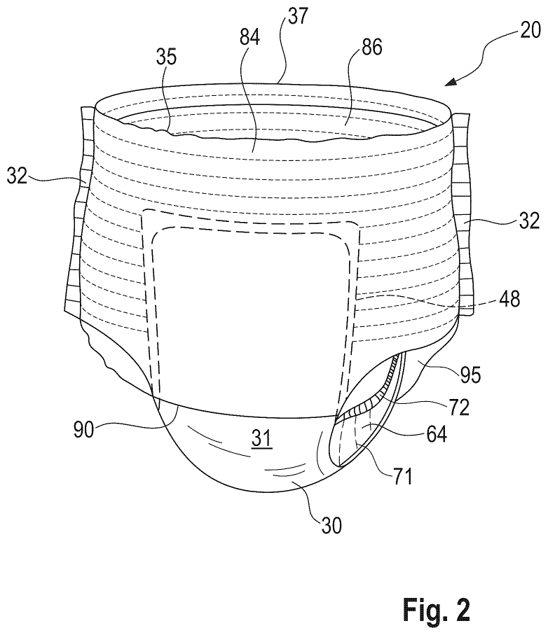

FIGS. 1 and 2 are a perspective view of an absorbent article 20 of the present invention. The absorbent article 20 has a longitudinal centerline L1 which also serves as the longitudinal axis, and a transverse centerline T1 which also serves as the transverse axis. While the longitudinal centerline L1 notionally divides the absorbent article into left and right halfes of equal width, the transverse centerline T1, extending through the crotch region of the absorbent article and being perpendicular to the longitudinal centerline L1, notionally divides the absorbent article into front and back halfs having equal length. The absorbent article 20 has a body-facing surface, a garment-facing surface, a front waist region 34 with a front waist edge 35 (the front waist region e.g. being a front elastic belt 84), a back waist region 36 with a back waist edge 37 (the back waist region e.g. being a back elastic belt 86), a crotch region 30 extending longitudinally between the front and back waist region 34, 36. The absorbent article may have side seams 32 which May join the front waist region and the back waist region (e.g., the front elastic belt 84 and the back elastic belt 86), to form two leg openings and a waist opening.

The absorbent article 20 may be a pant with a ring-like elastic belt 40 (herein referred to as “belt-type pant”) comprising an absorbent main body 38 to cover the crotch region 30 of the wearer, a front elastic belt 84 and a back elastic belt 86. The absorbent main body 38 extends into and overlaps with the front and back elastic belt 84, 86. The front and back elastic belts 84, 86 may form a discrete ring-like elastic belt 40 extending transversely defining the waist opening and being discontinuous in the longitudinal direction. For the belt-type pant, the discrete ring-like elastic belt 40 may also be referred to as the elastic belt 40. For the belt-type pant as exemplified in FIGS. 1 and 2, the front and back elastic belts 84, 86 and the absorbent main body 38 jointly define the leg openings.

For a belt-type pant, the front elastic belt 84 is the front waist region 34, and the back elastic belt 86 is the back waist region 36, and the remainder is the crotch region 30.

While not shown, the absorbent article 20 may be a so-called uni-body type pant configured such that the outer cover layer of the absorbent main body 38 and the garment facing layer of the elastic belt 40 are common. For the uni-body type pant, the portion extending in the transverse direction between the side seams 32, respectively, are considered the front waist region 34 and the back waist region 36, and the remainder is the crotch region 30. For the uni-body type pant, the front waist region 34 is considered the front elastic belt 84, and the back waist region 36 is considered the back elastic belt 86.

The absorbent article 20 comprises a topsheet 24, a backsheet 26 and an absorbent core disposed between the topsheet and the backsheet. The topsheet, backsheet and absorbent core may jointly be referred to as ‘absorbent main body’ 38. The absorbent main body 38 may further comprise an outer cover layer 42 for covering the garment-facing side of the backsheet 26. The topsheet 24 may be a water permeable substrate. The backsheet 26 may be a water impermeable film. The outer cover layer 42 may be a nonwoven web. The absorbent main body 38 comprises an absorbent core for absorbing and containing body exudates disposed on the absorbent main body 38. The topsheet 24 and backsheet 26 may be have a larger longitudinal and transverse dimension than the absorbent core, and the absorbent core may be surrounded by the topsheet, and the backsheet. The absorbent main body 38 may have a generally rectangular shape, left and right longitudinally extending side edges 48 and front and back transversely extending end edges 50. The absorbent core may exist through the entire longitudinal dimension of the crotch region and extend partly in the front and back waist regions 34, 36. The absorbent main body 38 may have a front waist panel 52 positioned in the front waist region 34 of the absorbent article 20, a back waist panel 54 positioned in the back waist region 36, and a crotch panel 56 between the front and back waist panels 52, 54 in the crotch region 30. The crotch panel 56 forms the crotch region 30. The center of the front elastic belt 84 may be joined to a front waist panel 52 of the absorbent main body 38, the center of the back elastic belt 86 may be joined to a back waist panel 54 of the absorbent main body 38. The front and back elastic belts 84, 86 may each have a left side panel and a right side panel 82 where the absorbent main body 38 does not overlap. The portion of the front and back elastic belts 84, 86 adjacent to the waist edge may also not overlap with the absorbent main body 38, i.e., the absorbent main body 38 does not longitudinally extend to the front and back waist edge. In a belt-type pant, the front elastic belt 84 is indirectly joined to the back elastic belt 86 along the longitudinal dimension by the absorbent main body 38.

The absorbent main body 38 comprises elasticized barrier leg cuffs 64 extending at least partially between the end edges 50 along each of the longitudinally extending side edges 48 of the absorbent main body 38 in the crotch region 30, and extending into the front and back waist region 34, 36.

The barrier leg cuffs each have a proximal edge 71 attached to the topsheet 24 and/or the backsheet and a free terminal edge 72, which is intended to contact and form a seal with the wearer's skin when the absorbent article is applied on a wearer. In use, at least in the crotch region 30, the leg cuffs are in active elasticity to provide gasketing around the leg openings. The free terminal edge 72 of each barrier leg cuff 64 is folded towards the longitudinal centerline L1 and onto the topsheet 24 when the absorbent article 20 is laid flat and stretched out, and, while laid flat, each of the barrier leg cuffs 64 overlays the topsheet 24 in the transverse direction. Each of the barrier leg cuffs extends from the proximal edge 71 to the free terminal edge 72 in the transverse direction of the absorbent article.

The barrier leg cuffs 64 may be made of material having high fluid impermeability. The barrier leg cuffs may be folded around the longitudinally extending side edges 48 of the absorbent main body, such that a portion of the barrier leg cuffs is attached to the garment-facing surface of the backsheet, and/or to the garment-facing surface of the optional outer cover layer.

The absorbent main body 38 may further comprise leg elastics. The barrier leg cuffs 64 may be positioned laterally inboard of leg elastics.

Each barrier leg cuff 64 may have the free terminal edges attached to the topsheet at and/or adjacent to the transversely extending end edges 50 of the absorbent main body 38. The free terminal edges, between the portions which are attached to the topsheet, may extend upwards from a body-facing surface of the absorbent main body 38 when the absorbent article is worn.

Each of the barrier leg cuffs 64 may comprise one or more elastic strands near and/or at the free terminal edge.

The absorbent core is intended and able to store the liquid that enters the absorbent article during use (thus generally having the by far hightest absorbent capacity) and that comprises an absorbent material. In some instances, absorbent material may be positioned within a so-called core bag or a core wrap, such as a nonwoven web (the core bag or core wrap being comprised by the absorbent core). The absorbent material may be profiled or not profiled, especially along the longitudinal centerline L1, but may also be profiled along the transverse centerline T1 depending on the specific absorbent article. “Profiled” means that the absorbent material is not homogeneously distributed across the surface area of the absorbent core. The absorbent core may comprise, consist essentially of, or consist of, a core wrap, and absorbent material enclosed within the core wrap, and possibly adhesive to immobilize the absorbent material. The absorbent material may, for example, comprise or consist of a) superabsorbent polymer material, such as superabsorbent polymer particles and/or superabsorbent polymer fibers, or b) a mixture of superabsorbent polymer particles and cellulose fibers, or c) only cellulose fibers, or d) a high internal phase emulsion foam, or e) combinations of any of a) to d). In some instances, the absorbent material may comprise at least 80%, at least 85%, at least 90%, at least 95%, at least 99%, or up to 100% superabsorbent polymers, by weight of the absorbent material. In such instances, the absorbent material may be free of cellulose fibers, or at least mostly free of cellulose fibers. The superabsorbent polymer material may be immobilized by adhesive, such as by adhesive fibers. The absorbent core periphery, which may be the periphery of the core wrap, may define any suitable shape, such as rectangular “T,” “Y,” “hour-glass,” or “dog-bone” shaped, for example. Preferably, the absorbent core has a rectangular shape. An absorbent core periphery having a generally “dog bone” or “hour-glass” shape may taper along its width towards the crotch region of the absorbent article.

The absorbent core may have areas with reduced caliper (wherein the term “reduced” includes areas free of the absorbent material of the absorbent core, e.g., areas where only the core wrap material is present). Areas with reduced caliper may be areas having little or no absorbent material, where a body-facing surface of the core bag may be joined to a garment-facing surface of the core bag. These areas having little or no absorbent material may be referred to as “channels”. These channels can embody any suitable shapes and any suitable number of channels may be provided. In other instances, though less preferred, the absorbent core may be embossed to create the impression of channels.

The absorbent article further comprises an acquisiton system 70. The absorbent core and the acquisiton system are sandwiched between the topsheet and the backsheet, with the acquisiton system being provided between the topsheet and the absorbent core. The acquisiton system comprises one or more layers.

The acquisition system may have a longitudinal dimension which is parallel to the longitudinal direction of the absorbent article. The acquisiton system 70 may be equally divided in three sections, the section being closest to the front waist edge 35 of the absorbent article 20 being the front section 75, the section being closest to the back waist edge 37 of the absorbent article 20 being the back section 76, and the section in between the front and back section being the center section 77. At least the front section 75 may extend underneath the barrier leg cuffs 64 when the barrier leg cuffs 64 are folded onto the topsheet 24.

When a wearer lies on her or his side, e.g., while sleeping, urine may flow over the topsheet towards the longitudinally extending side edges of the absorbent article. This has been found to be especially likely for the longitudinally extending side edges in the front waist region of the article (e.g., in the belt area adjacent to the side seams of an absorbent pant). Also, for wearers laying on their belly, there is a risk that urine flows over the topsheet towards the longitudinally extending side edges in the front waist region of the article. The absorbent core is often too slow to absorb larger amounts of urine fast enough before leakage through the longitudinally extending side edges occurs. Moreover, absorbent cores often have relatively low amounts of absorbent material at their longitudinally extending side edges, especially in the front waist region. Thus, urine that has flown towards this region cannot be readily absorbed, promoting leakage. Acquisition systems are oftentimes not as wide as the absorbent core, i.e., they do not extend towards the longitudinally extending side edges of the absorbent core, thereby further increasing the risk of leakage. Consequently, having the front section of the acquisiton system at least partly extending underneath the barrier leg cuffs can help to reduce the risk of leakage. The acquisiton system is able to quickly absorb the urine and can transport it subsequently to the absorbent core. Thereby, the acquisition system provides void volume and acts as a temporary reservoir for the urine.

The acquisition system may comprise one or more layer(s). The transverse dimension of at least one layer may be wider in the front section 75 than in the center section 77 of the at least one layer (and of the acuqision system as a whole, i.e. with all layers, if more than one layer is present). This provides sufficient void volume towards and in the front waist area adjacent the longitudinally extending side edges. At the same time, bulk in the crotch area is not unnecessarily increased, which is beneficial for wearer comfort and allows economical use of the acquisition material. The transverse dimension of at least one layer may be wider in the front section 75 than in the back section 76 of the at least one layer (and of the acquision system as a whole, i.e. with all layers, if more than one layer is present). The transverse dimension of at least one layer may be wider in the center section 77 than in the back section 76 of the at least one layer (and of the acuqision system as a whole, i.e. with all layers, if more than one layer is present).

If the respective layer has varying width in the front section 75, in the center section 77 and/or in the back section 76, the extension in the transverse direction in that section is the average extension in the transverse direction in the respective section.

The acquisition system may consist of two layers. Alternatively, the acquisiton system may comprise more than two layers, e.g. the acquisition system may consist of three layers or of four layers.

If the acquisition system comprises more than one layer, at least the layer of the acquisition system 70 which has the highest basis weight of all layers of the acquisition system 70, may have a transverse dimension which is wider in the front section 75 than in the center section 77 and in the back section 76.

The transverse dimension of at least one layer of the acquisition system in the front section 75 of the acquisition system 70, e.g. the layer having the highest basis weight, may be at least 10%, or at least 15%, or at least 20% wider in the front waist region 34 than in the center section 77 and in the back section 76.

The average basis weight of the front section 75 of the acquisition system may be higher than the average basis weight of the center section 77 and of the back section 76.

The average basis weight of the front section 75 of the acquisition system may be at least 20%, or at least 25%, or at least 30%, or at least 35% higher in the front section 75 than the average basis weight of the center section 77 and of the back section 76.

The acquisition system 70 may have a transverse dimension which is parallel to the transverse direction of the absorbent article,

The front section of the acquisition system may be equally divided in three portions (i.e., each portion having the same transverse dimension), namely two side portions 78 and one center portion 79 in between the two side portions. The basis weight may be higher in each of the two side portions than in the center portion.

The acquisition system, which is provided between the topsheet and the absorbent core, may comprises hydrophilic materials that provide significant wicking of bodily exudates. These materials may dewater the topsheet and quickly move bodily exudates into the absorbent core. The acquisition materials of the acquisition system may comprise one or more nonwoven materials, foams, cellulosic materials, cross-linked cellulosic materials, air laid cel-lulosic nonwoven materials, spunlace materials, or combinations thereof, for example. Typically, the one or more layers of the acquisition system may each have a width and length that are smaller than the width and length of the topsheet.

The first layer of the acquisition system may comprise a nonwoven material and as second layer of the acquisition system may comprise a cross-linked cellulosic material. The second layer of the acquisition system may be provided between the first layer of the acquisition system and the absorbent core. The first layer of the acquisition system may be provided between the topsheet and the second layer of the acquisition system. The second layer, e.g., the layer comprising or consisting of cross-linked cellulosic material, may have a higher basis weight that the first layer, e.g., the layer made of a nonwoven material.

If the acquisition system—or one or more of its layers—has varying basis weight in the various sections (i.e. front, center and back section) and/or portions (i.e., the two side portions and the center portion) of the acquisition system, the basis weight referred to herein above is the average basis weight in the respective section and/or portion.

The first layer of the acquisition system may be in direct contact with the topsheet and the second layer of the acquisition system may be in direct contact with the absorbent core.

The acquisition system may serve as a temporary reservoir for liquid until the absorbent core can absorb and store the liquid, and for subsequent distribution of the liquid into the absorbent core in an efficient manner.

The acquisition system may be free of superabsorbent polymer. The acquisition system may also be free of unmodified cellulose fibers.

The function of a second layer of the acquisition system (provided between the first layer of the acquisition layer and the absorbent core) is typically to spread the insulting fluid liquid over a larger surface within the absorbent article so that the absorbent capacity of the absorbent core can be more efficiently used. The second layer of the acquisition system may be made of a nonwoven web based on synthetic or cellulosic fibers and having a relatively low density. The second layer of the acquisition system may typically have an average basis weight of from 30 to 400 g/m², in particular from 80 to 300 g/m². The second layer of the acquisition system may not be formed of a coherent, self-sustaining web or sheet but may be a layer with little integrity on its own.

The second layer of the acquisition system may for example comprise at least 50%, or 60%, or 70%, or 80%, or 90% by weight of cross-linked cellulose fibers. The cross-linked cellulosic fibers may be crimped, twisted, or curled, or a combination thereof including crimped, twisted, and curled. The cross-linked cellulosic fibers provide higher resilience compared to non-modified cellulose fibers and therefore higher resistance against the compression in the product packaging or in use conditions of the absorbent article, e.g. under baby weight. This provides the second layer of the acquisition system with a relatively high void volume, permeability and liquid absorption, and hence reduced leakage and improved dryness.

The second layer of the acquisition system comprising cross-linked cellulose fibers, may comprise other fibers, but this layer may advantageously comprise at least 50%, or 60%, or 70%, or 80%, or 90% or even up to 100%, by weight of the layer, of cross-linked cellulose fibers. Examples of such mixed layer of cross-linked cellulose fibers may comprise 70% by weight of chemically cross-linked cellulose fibers, 10% by weight polyester (PET) fibers, and 20% by weight untreated pulp fibers. In another example, the layer of cross-linked cellulose fibers may comprise 70% by weight chemically cross-linked cellulose fibers, 20% by weight lyocell fibers, and 10% by weight PET fibers. In another example, the layer may comprise 68% by weight chemically cross-linked cellulose fibers, 16% by weight untreated pulp fibers, and 16% by weight PET fibers.

The acquisition system may further comprise a first layer (provided between the second layer of the acquisition system and the topsheet), whose function is typically to quickly acquire the fluid away from the topsheet so as to provide a good dryness for the wearer. The first layer of the acquisition system is typically placed directly under the topsheet and directly above the second layer of the acquisition system. The first layer of the acquisition system may typically be or comprise a non-woven web, for example a SMS or SMMS material, comprising two outer spunbonded(S) layers with one or more melt-blown (M) layers in between, or alternatively a carded nonwoven web comprising a binder to consolidate the fibers and provide web integrity. The nonwoven web may, in particular, be latex binder bonded. Exemplary first layers for the acquisition system are disclosed in U.S. Pat. No. 7,786,341. Carded, resin-bonded nonwoven webs may be used, in particular where the fibers used are solid round or round and hollow PET staple fibers (such as a 50/50 or 40/60 mix of 6 denier and 9 denier fibers).

A carded resin-bonded first layer of the acquisition system may be stabilized by a latex binder, for example a styrene-butadiene latex binder (SB latex). Processes for obtaining such lattices are known, for example, from EP 149 880 (Kwok) and US 2003/0105190 (Diehl et al.). The binder may be present in the first layer of the acquisition system in excess of 15%, or of 20% by weight, but may be present by not more than 40%, or not more than 35% by weight of the upper layer. SB latex is, for example, available under the trade name GENFLO™ 3160 (OMNOVA Solutions Inc.; Akron, Ohio).

The elastic belt 40 of the absorbent article of the present invention acts to dynamically create fitment forces and to distribute the forces dynamically generated during wear. The front and back elastic belts 84, 86 may be joined with each other only at the side edges 89 to form side seams 32, a waist opening and two leg openings (such that the waist opening is formed by the front waist edge and the back waist edge of the absorbent article). Each leg opening may be provided with elasticity around the perimeter of the leg opening. The elasticity around the leg opening may be provided by the combination of elasticity from the front elastic belt 84, the back elastic belt 86, and the barrier leg cuffs 64.

The longitudinal length of the backsheet 26 and the outer cover layer 42 may be the same, or may be varied. For example, the outer cover layer 42 may have a shorter length compared to that of the backsheet 26, such that the outer cover layer 42 is devoid where the absorbent main body 38 overlaps the elastic belt 40. By such configuration, the elastic belt may have better breathability. Further, such configuration may provide cost saving. The transverse width of the backsheet and the outer cover layer 42 may be the same, or may be varied. For example, the backsheet may have a shorter transverse width compared to that of the outer cover layer 42. By such configuration, the longitudinal side edges 48 of the crotch panel 56, which make part of the leg openings, may have better breathability. Further, such configuration may provide cost saving.

For the belt-type pant, the longitudinal length LB of the back elastic belt 86 and the longitudinal length LF of the front elastic belt 84 may be provided the same, or the back elastic belt 86 may have a greater longitudinal length LB. When the absorbent article is assembled to form the waist opening and the leg openings, the absorbent article 20 is folded along the transverse centerline T1 such that the front distal edge 88 is aligned with the back distal edge 88 (the front distal edge being the front waist edge 35 and the back distal edge being the back waist edge 37). The front side edge 89 is also aligned with a portion of the back side edge 89. Then the front elastic belt 84 and the back elastic belt 86 are joined at the front and back side edges 89 at the scams 32. The front and back proximal edges 90 of the front and back elastic belt 84, 86, however, may not be aligned to one another. The back proximal edge 90 of the back elastic belt 86 may be disposed longitudinally closer than the front proximal edge 90 of the front elastic belt 84 relative to the transverse centerline T1 such that the proximal portion of the back side panel 82 extends toward the crotch panel 56 of the main body 38 beyond the front proximal edge 90. The side edge of the proximal portion of the back side panel 82 may not be joined to anywhere and free from attachment. Thus, the proximal portion of the back side panel 82 provides a buttock cover 95. In such embodiments, the front waist region 34 is smaller than the back waist region 36 along the longitudinal dimension of the absorbent article.

The front elastic belt 84 and back elastic belt 86 are configured to impart elasticity to the belt 40. The front elastic belt 84 and the back elastic belt 86 may each comprise a laminate, the laminate comprising a plurality of elastic members 96F, 96S, such as elastic strands, running in the transverse direction, an inner sheet 94, an outer sheet 92. The back elastic belt (and also the front elastic belt) may comprise an outer sheet fold 931 which is an extension of the outer sheet 92 and is formed by folding an extension 93 of the outer sheet 92 material at the distal edge 88 of the front and back elastic belts 82, 84; wherein the belt elastic members 96F, 96S, such as elastic strands, are sandwiched between the inner and outer sheet 94, 92. The front elastic belt 84 and the back elastic belt 86 may each be made only by elastic members 96S, such as elastic strands, the inner sheet 94, the outer sheet 92, and the outer sheet fold 931, and, optionally, by adhesive to attach the inner and outer sheet to each other and/or to attach the elastic strands to the inner and/or outer sheet. The belt elastic members 96F, 96S, such as elastic strands, may extend in the transverse direction to provide a ring like elastic belt 40 when the front elastic belt 84 and the back elastic belt 86 are joined along side seams 32. At least some of the elastic members 96F, 96S, such as elastic strands, extend in the transverse direction substantially parallel to each other. All of the elastic members 96F, 96S, such as elastic strands, may extend in the transverse direction substantially parallel to each other. Such an article may be economically made. The front and back elastic belt 84, 86 each may have transversely continuous proximal and distal edges, the proximal edge 90 being located closer than the distal edge 88 relative to the longitudinal center of the article. At least 10%, or at least from about 15% to not more than about 70%, of the front and back elastic belts from the waist opening in the longitudinal direction may be a laminate in active elasticity along the entire transverse dimension LW of the front and back elastic belts 84, 86. For each front and back elastic belt 84, 86, the region overlapping the front and/or back waist panel 52, 54 of the absorbent main body 38 may be removed of its elastic activity. Such region removed of elastic activity is referred herein as the “elastic cut window”, and the remainder of the intact elastic member capable of imparting elasticity is defined as the “effective length of elasticity of an elastic member”.

The tensile stress (N/m) of the entirety of the front and back elastic belts 84, 86, respectively, may be profiled in order to provide the functional benefits of the present invention, such as case of stretch and application, while also maintaining certain force during wear, to prevent the article from sagging after loading. When the elasticity of the front and back elastic belts 84, 86 are provided by a plurality of elastic members 96F, 96S, such as elastic strands, running in the transverse direction, the tensile stress may be adjusted by one or more of the following methods; 1) elongation rate of the elastic member 96F, 96S 2) density (dtex) of the elastic member 96F, 96S; 3) longitudinal pitch of multiple elastic members 96F, 96S; and 4) effective length of elasticity of the elastic member 96F, 96S in the transverse direction. By elongation, “0% elongation” is meant the original length of the elastic member.

Alternatively to being a pant, the absorbent article may be a so-called taped diaper, i.e., an absorbent article provided with a fastening system to close the absorbent article around the waist of the wearer. A taped diaper may comprise back cars in the back waist region. The back cars may comprise fasteners and may extend from the back waist region of the absorbent article and attach (using the fasteners) to the landing zone area or landing zone material on a garment-facing portion of the front waist region of the absorbent article. The absorbent article may also have front cars in the front waist region.

The landing zone area may be in the back waist region if the absorbent article fastens from front to back or, preferably, may be in the front waist region if the absorbent article fastens back to front. In some instances, the landing zone may be or may comprise one or more discrete nonwoven materials that are attached to a portion of the garment-facing surface (e.g., on an outer cover nonwoven) of the front waist region or of the back waist region, depending upon whether the absorbent article fastens in the front or the back. In essence, the landing zone is configured to receive the fasteners and may comprise, for example, a plurality of loops configured to be engaged with, a plurality of hooks on the fasteners, or vice versa.

The absorbent article in the form of a taped diaper may have front and/or back cars. Only one set of cars may be required in most taped diapers. The single set of cars may comprise fasteners 46 configured to engage the landing zone or landing zone area. If two sets of cars are provided, in most instances, only one set of the cars may have fasteners (preferably the back cars), with the other set being free of fasteners. The cars, or portions thereof, may be elastic or may have elastic panels. In an example, an elastic film or elastic strands may be positioned intermediate a first nonwoven material and a second nonwoven material. The elastic film may or may not be apertured. The cars may be shaped. The cars may be integral (e.g., extension of the outer cover material, the backsheet, and/or the topsheet) or may be discrete components attached to absorbent main body on a wearer-facing surface, on the garment-facing surface, or intermediate the two surfaces.

### Waist Guard

The absorbent article of the present invention comprises a front waist guard FWG, which is disposed in and attached to the front waist region 34 such that a front pocket 203 is formed. The front pocket 203 extends along the longitudinal direction from a closed base line 205 at or adjacent to the front waist edge 35 towards an open edge 206. The dimension from the closed base line 205 to the open edge 206 parallel to the longitudinal centerline L1 defines the front pocket length FPL. If the front pocket has varying length (i.e., varying longitudinal dimension) across its width (i.e., across its transverse dimension), the front pocket length FPL is the maximum length. The closed base line 205 may be longitudinally spaced from the open edge 206, such that the open edge 206 may be closer to the transverse centerline T1 of the absorbent article 20 than the closed base line 205. The complete area of the first pocket 203 may superpose the backsheet 26.

The acquisition system comprised by the absorbent main body is provided in the front pocket 203 along at least 10% or at least 20%, or at least 30% of the front pocket length FPL. The acquisition system comprised by the absorbent main body may be provided in the front pocket 203 along not more than 80% or not more than 75%, or not more than 70% of the front pocket length FPL.

While waist guards in the front waist region may be generally known, it has now been found that they may not be able to effectively reduce leakage through the front waist edge. For wearers laying on their belly, the absorbent article may not be able to readily absorb urine flowing over the topsheet towards the front waist region and underneath the front waist guard. The absorbent core often has reduced amounts of absorbent material in the front waist region near the front waist edge. Also, the acquisition system typically is shorter than the absorbent core, which has been found to further enhance the potential for leakage through the front waist edge. Hence, it has been found that it is beneficial to have the acquisiton system extending underneath the front waist guard to enable fast acquisition of free urine.

The absorbent article may further comprise a back waist guard BWG disposed in and attached to the back waist region 36 such that a back pocket is formed, the back pocket extending along the longitudinal direction from a back closed base line at or adjacent to the back waist edge 37 towards a back open edge of the back pocket.

In absorbent articles in the form of a belt-type pant, the front waist guard FWG may be partially bonded to the front elastic belt 84, such that the bonded portion of the front waist guard FWG superposes the backsheet 26 by at least 3 mm, preferably by at least 5 mm, more preferably by at least 10 mm along each of left and right side edges of the pocket 203, thereby defining the left and right side edges of the pocket 203.

In belt-type pants, the front elastic belt 84 and the back elastic belt 86 may each comprise an inner sheet 94 and an outer sheet 92, and the waist guard may be an extension 93 of the outer sheet 92 of the front elastic belt 84. The extension 93 may be folded over inwardly to a body-facing surface of the absorbent article, such that the fold line of the extension forms the front waist edge 36 of the absorbent article 20.

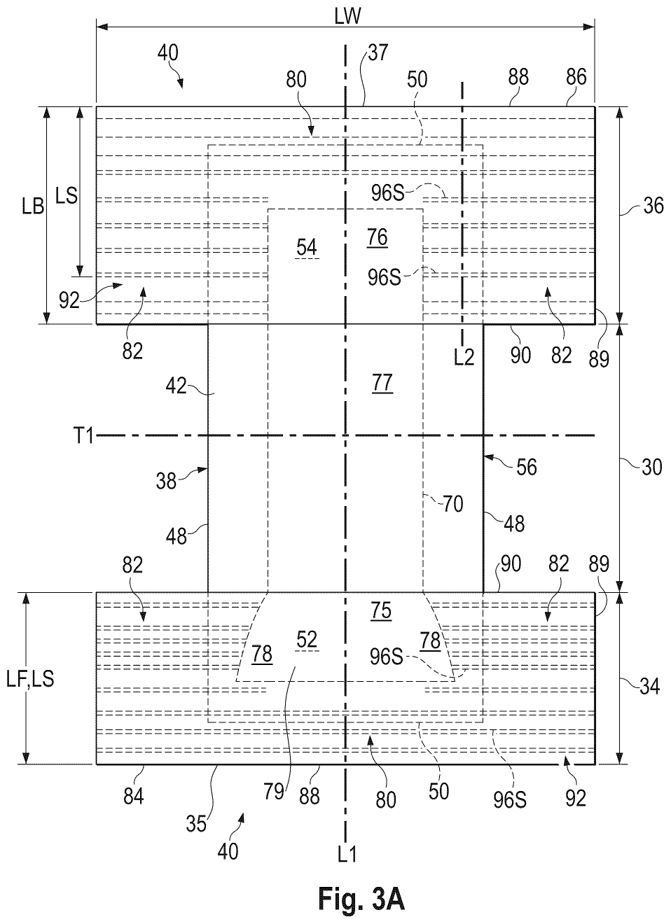

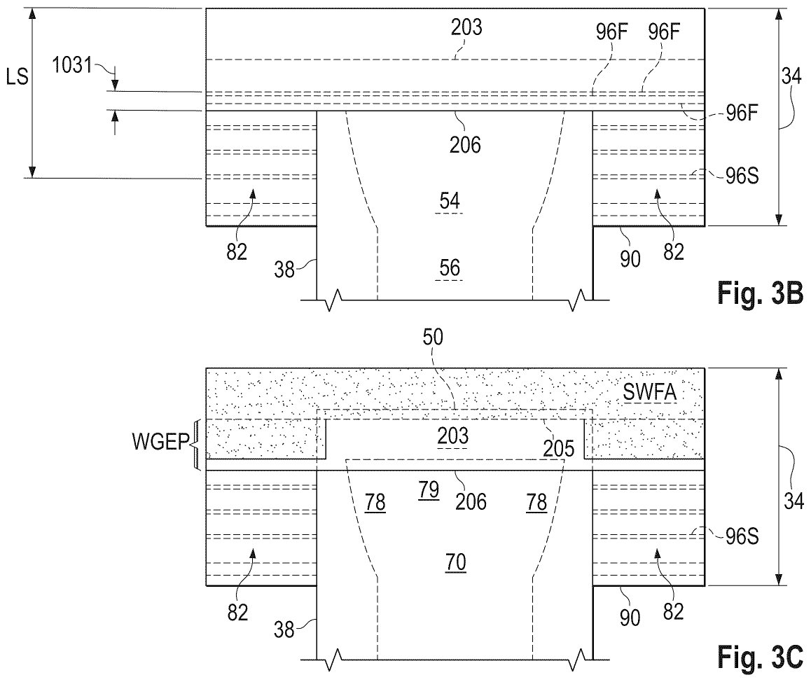

As exemplified in FIGS. 3A and 3B, the front and back elastic belts 84, 86 may each be divided into multiple zones spanning in the transverse direction and defined by its location from the distal edge 88 to the proximal edge 90 relative to the percentage of the seam length LS wherein the distal edge 88 is considered 0% and the proximal edge 90 is considered 100%. The multiple zones may be configured to provide different tensile stress, or different functions to the front and back elastic belts 84, 86, respectively. In the absorbent article of the present invention, the front waist guard FWG may be disposed within a location of 5-60% of LS on the front elastic belts 84. The complete front waist guard FWG may be provided within the 5% to 60% of LS of the front elastic belt 84. The front waist guard FWG may be shorter, so it does not need to span the entire 5-60% of LS as long as the complete front waist guard FWG is provided within such dimension. The front waist guard FWG may be positioned at a dimension of from from about 5% to about 55% of LS, or from about 5% to about 50% of LS, or from about 10% to about 55% of LS, or from about 10% to about 50% of LS in the front elastic belt 84.

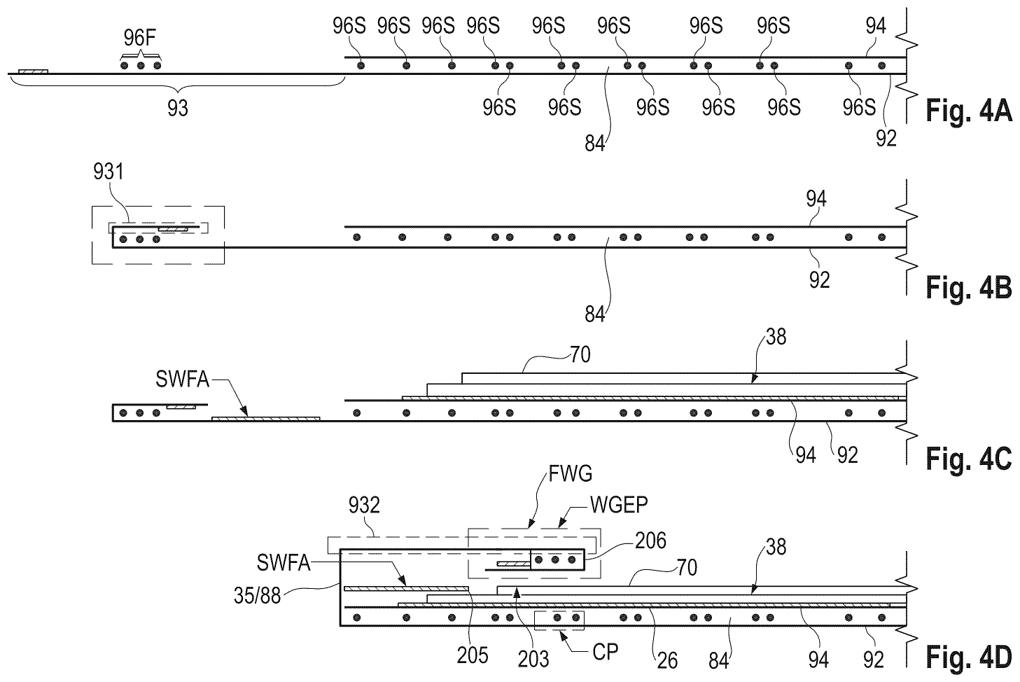

Referring to FIGS. 3B, 3C, and 4A-4D, the front waist guard FWG may be made by an extension of the outer sheet 92 of the front elastic belt extending beyond the longitudinal dimension of the inner sheet 94, which extension of the outer sheet 92 may be folded inwardly 2 times in order to encompass a first elastic member 96F and further to provide the front waist guard FWG in the intended position relative to the side seam length LS of the front elastic belt. As a result of the 2 folds, the first elastic member 96F is sandwiched between the first outer sheet fold 931 and the second outer sheet fold 932, wherein the first outer sheet fold 931 constitutes the garment facing side of the front waist guard FWG, and the second outer sheet fold 932 constitutes the body facing surface. Referring to FIGS. 3C and 4D, a certain longitudinal dimension from the distal edge of the second outer sheet fold 932 may be bonded to the remainder of the front elastic belt, such bonding shown as SWFA, however, leaving some longitudinal dimension unbonded. The position provided most distal of the unbonded portion is the closed base line 205 of the front waist guard, and the common proximal edge of the first outer sheet fold 931 and second outer sheet fold 932 is the open edge 206 of the front waist guard. The front waist guard FWG is defined as having a longitudinal dimension spanning from the closed base line 205 and the open edge 206, and having a transverse dimension matching the width of the front elastic belt to which it is provided. The left and right transverse edges of the front waist guard FWG may also be bonded to the remainder of the front elastic belt belt, such bonding shown as SWFA. Preferably the bonding is made by adhesive The unbonded portion thus provided is the front pocket 203. The bondings provided to define the area of the front pocket 203 may be so configured to provide the front pocket 203 substantially superposing the backsheet, preferably completely superposing the backsheet in both in the longitudinal dimension parallel to the longitudinal centerline L1 of the article as well as in the transverse direction parallel to the transverse centerline T1 of the article.

The bonded portion (shown as SWFA) of the front waist guard may superpose the backsheet by at least 5 mm, or by at least 10 mm along each of the left and right side edges of the front pocket, wherein the respective part of the edge of the bonded portion may delimit and thus define the left and right side edges of the front pocket. Moreover, the bonded portion (shown as SWFA) may superpose the backsheet by at least 5 mm, or by at least 10 mm along the closed base line of the front pocket, wherein the respective part of the edge of the bonded portion may delimit and thus define the closed base line of the front pocket.

Referring to FIG. 3B, the first elastic member 96F provides the front waist guard elastic portion WGEP, wherein elasticity may be imparted by one or more elastic strands, elastic ribbons, mechanically activated nonwovens, or otherwise. The front waist guard elastic portion WGEP is defined as having a longitudinal dimension 1031 spanning from the open edge 206 to the distal edge of the first elastic member 96F. When the first elastic member 96F is a plurality of elastic strands, the distal edge of the front waist guard elastic portion WGEP is the position of the elastic strand which is positioned most distal. There may be 2-10 elastic strands, or from 2-5 elastic strands, or 2-8 elastic strands, or 3-4 elastic strands, or 3-8 elastic strands, or 4-6 elastic strands disposed on the front waist guard elastic portion. The longitudinal dimension of the front waist guard elastic portion WGEP is smaller than that of the front waist guard WG. The longitudinal dimension of the front waist guard elastic portion WGEP may be no greater than about 40 mm, or no greater than about 35 mm, or no greater than about 30 mm, or no greater than about 25 mm, or from 15 mm to about 40 mm, or from about 10 mm to about 35 mm, or from about 10 mm to about 25 mm. As exemplified in FIGS. 3B and 3C, the front pocket may have a rectangular shape, however, the front pocket may take other shapes by changing the bonding area SWFA such that the closed base line 205 of the front waist guard is shaped. For example, the closed base line 205 may be concave or convex toward the pocket to match the wearer's anatomy. The closed base line 205 of the front waist guard portion may also be straight and parallel to the distal edge of the front elastic belt 84. The longitudinal dimension (=the length=of the pocket is defined as the longest distance of the front pocket extending parallel to the longitudinal centerline L1 of the article from the closed base line to the open edge. The longitudinal dimension of the front pocket may be at least 25 mm, or at least 30 mm, or at least 35 mm, mm. The longitudinal dimension of the front pocket is not more than 120 mm, or may be not more than 100 mm, or may be not more than 90 mm. For a front pocket having a closed base line which is are not straight but, for example, concave or covex, the shortest distance between the closed base line to the open edge may be at least 15 mm, or may be at least 20 mm.

The transverse dimension of the front pocket is defined as the longest distance of the front pocket extending parallel to the transverse centerline T1 of the article from the left side edge of the front pocket to the right side edge of the front pocket. The left and right side edges extend between the closed base line and the open edge of the front pocket. The transverse dimension (=the width) of the front pocket of the front waist guard may be from 40% to 96%, or from 50% to 95%, or from 60% to 95% of the transverse dimension of the transversely extending edge of the backsheet, which is provided in the front waist region. The transversely extending edge of the backsheet may extend parallel to the transverse centerline T1 of the absorbent article.

Referring to FIGS. 3B and 4D, the remainder of the belt superposing the front waist guard elastic portion WGEP is defined a correlated portion CP. Namely, the correlated portion CP has the same longitudinal dimension 1031 as the front waist guard elastic portion WGEP. The correlated portion CP may be elasticized or may not be elasticized, wherein the tensile stress of the front waist guard elastic portion WGEP is higher than that of the correlated portion CP. The correlated portion CP may be elasticized wherein the tensile stress of the front waist guard elastic portion WGEP is at least about 25% higher, or at least about 100% higher, preferably 200% higher than that of the correlated portion CP. Without being bound by theory, such tensile stress difference enables the front waist guard elastic portion WGEP to be in close contact with the wearer, while pulling away the open edge 206 from the correlated portion CP, thus opening the front pocket 203 towards the proximal side. Thus, the front pocket is configured to have good containment capacity. The correlated portion CP may have an elastic cut window such that some portion is elasticized, while the transverse center does not interfere with the opening of the front pocket. When the front waist guard elastic portion WGEP is elasticized by disposing a plurality of elastic strands 96F, one elastic strand is disposed from no less than 2 mm to 5 mm away from the open edge 103M. Placement of the elastic strand on the very edge of the open edge may be avoided in order to prevent the front waist guard causing irritation to the wearer. The waist guard elastic portion WGEP may be disposed with a plurality of elastic strands 96F with a relatively small pitch between each other. The elastic strands 96F of the front waist guard elastic portion WGEP may be disposed at a pitch of no more than about 12 mm, or no more than about 10 mm, or no more than about 6 mm, or between about 2 mm to about 8 mm, or between about 3 mm to about 9 mm, or between about 2 mm to about 6 mm.

The elastic strands 96F for providing the front waist guard elastic portion WGEP may be provided in color such that the existence and function of the front waist guard FWG is clearly communicated to the wearer or caregiver. At least 2 of the elastic strands for providing the front waist guard elastic portion WGEP may have a color which is visible from the body facing side of the article. The color may be selected from green, blue, purple, or a combination thereof, in order to be easily recognized, while not being confused with the color of containment. The same or similar color may be used for other elastic strands 96S disposed on the belt to provide a coordinated appearance.

The front waist guard FWG may be configured in a certain way relative to the absorbent main body to provide improved function. As mentioned above, the area of the front pocket 203 may be so configured to provide the front pocket substantially superposing the backsheet, preferably completely superposing the backsheet. Referring to FIGS. 3C and 4D, the longitudinal edges of the absorbent main body 38 may be positioned and bonded between the front waist guard FWG and the remainder of the front elastic belt. This is to avoid having sharp edges of the absorbent main body 38 on the body facing surface, and also to avoid leakage from the edges.

The front waist guard FWG may be configured in a certain way relative to the barrier leg cuffs 64 disposed along both transverse edges of the absorbent main body 38, to provide improved function. For the belt comprising the front waist guard FWG, the leg cuffs 64 may be in active elasticity at the point intersecting the open edge 206. By providing the leg cuffs in active elasticity in such position, the combined elasticity from the leg cuffs and front waist guard elastic portion WGEP may help hold the leg cuffs 64 and front waist guard FWG in close contact with the wearer, and further provide additional containment capacity for the front pocket 203.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article having a longitudinal centerline and a longitudinal direction along and in parallel to the longitudinal centerline; a transverse centerline and a transverse direction along and in parallel to the transverse centerline, a front waist region with a front waist edge, a back waist region with a back waist edge and a crotch region extending longitudinally between the front and back waist region, the absorbent article comprising:
an absorbent main body with longitudinally extending side edges and transversely extending end edges, the absorbent main body comprising a topsheet, a backsheet, an absorbent core, and an acquisition system, wherein the absorbent core and the acquisition system are sandwiched between the topsheet and the backsheet with the acquisiton system being provided between the topsheet and the absorbent core;
the absorbent main body further comprising elasticized barrier leg cuffs extending at least partially between the end edges along both longitudinally extending side edges of the absorbent main body in the crotch region, and extending into the front and back waist region,
the barrier leg cuffs each having a proximal edge attached to the topsheet and/or the backsheet and a free terminal edge, which is intended to contact and form a seal with the wearer's skin,
wherein a front waist guard is disposed in and attached to the front waist region such that a front pocket is formed, the front pocket extending along the longitudinal direction from a closed base line at or adjacent to the front waist edge towards an open edge, wherein the maximum dimension from the closed base line to the open edge parallel to the longitudinal centerline defines the front pocket length (FPL), wherein the acquisition system is provided in the front pocket along at least 10% of the front pocket length (FPL).

2. The absorbent article whereof claim 1, wherein the free terminal edge of each barrier leg cuff is folded towards the longitudinal centerline and onto the topsheet when the absorbent article is laid flat and stretched out, and each of the barrier leg cuffs overlays the topsheet in the transverse direction, extending from the proximal edge to the free terminal edge of each barrier leg cuff, and
wherein the acquisition system has a longitudinal dimension which is parallel to the longitudinal direction of the absorbent article, and which is equally divided in three sections, the section being closest to the front waist edge of the absorbent article being the front section, the section being closest to the back waist edge of the absorbent article being the back section, and the section in between the front and back section being the center section, wherein at least the front section extends underneath the barrier leg cuffs when the barrier leg cuffs are folded onto the topsheet.

3. The absorbent article of claim 1, wherein each barrier leg cuff has the free terminal edges attached to the topsheet at and/or adjacent to the transversely extending end edges of the absorbent main body and wherein the free terminal edges, between the portions which are attached to the topsheet, extend upwards from a body-facing surface of the absorbent main body when the absorbent article is worn.

4. The absorbent article of claim 1, wherein the barrier leg cuffs each comprise one or more elastic strands near and/or at the free terminal edge.

5. The absorbent article of claim 1, wherein the acquisition system comprises one or more than one layer, and wherein the transverse dimension of at least one layer is wider in the front section than in the center section an in the back section.

6. The absorbent article of claim 1, wherein the acquisition system comprises at least two layers, and wherein at least the layer of the acquisition system which has the highest basis weight of all layers of the acquisition system, has a transverse dimension which is wider in the front section than in the center section and in the back section.

7. The absorbent article of claim 1, wherein the transverse dimension of at least one layer in the front section of the acquisition system is at least 10% wider in the front waist region than in the center section and in the back section.

8. The absorbent article of claim 1, wherein the average basis weight of the front section is higher than the average basis weight of the center section and of the back section.

9. The absorbent article of claim 1, wherein the average basis weight of the front section is at least 20% higher in the front section than the average basis weight of the center section and of the back section.

10. The absorbent article of claim 1, wherein the front section of the acquisition system has a transverse dimension which is parallel to the transverse direction of the absorbent article, and which is equally divided in three portions, the portion, the three portions being two side portions and one center portion in between the two side portions, wherein the basis weight is higher in each of the two side portions than in the center portion.

11. The absorbent article of claim 1, wherein the longitudinal dimension of the pocket defined as the longest distance between the closed base line and the open edge is from about 25 mm to about 120 mm.

12. The absorbent article of claim 1, wherein the transverse dimension of the pocket is from 40 mm to 200 mm.

13. The absorbent article of claim 1, further comprising a back waist guard disposed in and attached to the back waist region such that a back pocket is formed, the back pocket extending along the longitudinal direction from a back closed base line at or adjacent to the back waist edge towards a back open edge of the back pocket.

14. The absorbent article of claim 1, wherein the absorbent article is an absorbent pant, wherein the absorbent pant comprises a front elastic belt provided as and defining the front waist region, a back elastic belt provided as and defining the back waist region, the front and back elastic belt being joined to each other along side seams, forming a waist opening, and a pair of leg openings, with the crotch region extending longitudinally between the front elastic belt and the back elastic belt;
the absorbent main body extending the entire longitudinal dimension of the crotch region and further extending partly into each of the front elastic belt and the back elastic belt.

15. The absorbent article of claim 14, wherein the pant is a belt-type pant with the front and back elastic belt forming a discrete elastic belt extending transversely to define the waist opening and being discontinuous in the longitudinal direction, wherein the discrete elastic belt and the absorbent main body jointly define the leg openings.

16. The absorbent article of claim 15, wherein the waist guard is partially bonded to the front elastic belt, such that the bonded portion of the front waist guard superposes the backsheet by at least 3 mm along each of left and right side edges of the pocket, thereby defining the left and right side edges of the pocket.

17. The absorbent article of claim 16, wherein the front elastic belt and the back elastic belt each comprise an inner sheet and an outer sheet, wherein the front waist guard is formed by an extension of the outer sheet of the front elastic belt, wherein the extension is folded over inwardly to a body-facing surface of the absorbent article, such that the fold line of the extension forms the front waist edge of the absorbent article.
